(19) United States
(12) Patent Application Publication
(10) Pub. No.: US 2010/0041128 A1
(43) Pub. Date: Feb. 18, 2010

(54) Microfluidic Device for Application of Shear Stress and Tensile Strain

(75) Inventors: Albert J. Banes, Hillsborough, NC (US); Jian Wang, Durham, NC (US); Michelle E. Wall, Apex, NC (US)

Correspondence Address: THE WEBB LAW FIRM, P.C., 700 KOPPERS BUILDING, 436 SEVENTH AVENUE, PITTSBURGH, PA, 15219, US

(73) Assignee: MedTrain Technologies, LLC, Hillsborough, NC (US)

(21) Appl. No.: 12/350,531

(22) Filed: Jan. 8, 2009

Related U.S. Application Data
(60) Provisional application No. 61/019,684, filed on Jan. 8, 2008.

(51) Int. Cl. C12M 3/00

(52) U.S. Cl. 435/287.9; 435/289.1; 435/294.1; 435/297.1

(57) ABSTRACT

The present invention provides a cell culture device for applying shear stress and strain on single cells. The device includes at least one channel, at least one cell culture chamber having at least one single-cell attachment surface, a flexible membrane, at least one vacuum channel, and an inlet and an outlet. The inventive single-cell culture assembly fluid flow for applying fluid induced stress and/or substrate induced stress to cultured cells.

The present application claims priority to U.S. Provisional Application No. 61/019,684, filed Jan. 8, 2008, which is incorporated herein by reference.

### BACKGROUND OF THE INVENTION

1. Field of the Invention

The present invention is directed toward a single-cell culture assembly used in the biomedical science field of tissue engineering and, more specifically, to a single-cell culture assembly through which fluid may flow for applying fluid induced stress and/or substrate induced stress to cultured cells.

2. Description of Related Art

Mechanotransduction is a term used to describe the ability of a cell to transduce a mechanical signal into biochemical signals. Culturing cells in a mechanically active environment better simulates the in vivo environment compared to standard tissue culture conditions since cells are normally subjected to multiple modes of deformation in vivo. Mechanical load has been found to stimulate growth, reorganize actin filaments, alter cell alignment, induce protein synthesis, and alter extracellular matrix protein expressions in a variety of cell types. The strain sensitive mechanisms that underlie these responses include ion channel activation, mechano-gated channels, integrin activation, second messengers, intercellular communication, and multiple phosphorylation pathways. Organelles such as the Golgi tendon organ, the Vater-Pacini corpuscle, and the motor plate ending are responsible for reception of strain and for transmission of signals through innervation to the brain. Some pathways may be dominant for mechanical load activation. However, it is likely that deformation stimuli share ligand-activated systems such as the receptor protein tyrosine kinase (RPTK) or jun activated kinase/JAK/STAT pathways cross-reactivity contributing to pathway activation, failsafe redundancy, signal amplification/dampening. and overall regulation and diversity in the systems. A load stimulus increases system strain from the basal state. As a cell responds to a load stimulus orienting to the strain field and polymerizing actin, the intrinsic strain field in the cell syncytium increases in intensity. Strain on individual cell-substratum (focal adhesions) or cell-cell contacts (ICAMs, gap junctions, other) may induce conformational changes that result in activation of signaling pathways. Signaling involves phosphorylation/phosphatase events dependent on kinases/phosphatases that activate/deactivate channel proteins, focal adhesion proteins, cytoplasmic filament proteins or receptor or nonreceptor protein tyrosine/serine/threonine kinases which elicit specific transcriptional and translational events. Two models for detection and response to mechanical deformation on the cellular and molecular levels have been well established: touch reception in C. elegans, and sound detection in the mammalian ear. These model systems are examples of outside-in signaling to external forces. The ability to further identify mechanosensitive genes will provide markers for gauging biomechanical robustness of tissue engineered constructs as well as identify potential gene targets for drug and gene therapy.

Systems exist for applying tensile and compressive strains. For instance, the FX-4000™ Tension Plus™ system (Flexcell International Corp., Hillsborough, N.C.) is a computer-regulated controller used for applying static and cyclic strain to cells cultured on rubber-bottomed culture plates or to cells seeded in collagen gels. Vacuum is used to deform the flexible bottomed membranes of culture plates over planar-faced loading posts applying tensile strain to the cells cultured on the membranes. Cylindrical shaped Loading Posts™ are used to produce equibiaxial strain and Arctangle™ (rectangle with curved short ends) posts are used to provide uniaxial strain (U.S. Pat. No. 6,472,202). The StageFlexer® (Flexcell International Corp., Hillsborough, N.C.) is a single well embodiment of the Tension Plus™ system. This compact strain device allows observation of cells under strain conditions in real time on a microscope stage. Similar to the Tension Plus™ system, strain is controlled in the StageFlexer® via computer regulated vacuum deformation of a silicone membrane over a loading post (U.S. Pat. No. 6,048,723). The Streamer® is a shear stress device that allows users to apply fluid shear stress to cells cultured in monolayer on special matrix coated Culture Slips®. A computer-controlled peristaltic pump regulates shear stress from 0 to 35 dynes/cm². When used with an OsciFlow® flow controller, the device can generate oscillating and pulsatile flow profiles. (U.S. Pat. No. 6,645,759). The FlexFlow™ is a shear stress device that allows the user to observe signaling responses to fluid flow or to strain before, during, or after applying a shear stress. Cells are cultured on matrix bonded rubber surfaces using StageFlexer® membranes or on matrix-treated glass Culture Slips®. Cells can be strained by a Tension Plus™ system before, during, or after applying shear stress. The system uses a computer controlled peristaltic pump to regulate shear stress from 0 to 35 dynes/cm² (U.S. Pat. No. 6,645,759).

Microfluidic devices provide a non-invasive method for continuously investigating cell behavior while allowing both spatial and temporal control of cell growth conditions, providing scientists with a tool to collect real-time data without disturbing cell culture conditions and enabling high-throughput analysis while decreasing time and costs. There are several custom designed microfluidic devices for cell-level experiments. However, there is currently no available microfluidic device for applying shear stress and strain simultaneously to single cells in vitro.

### SUMMARY OF THE INVENTION

In one embodiment, the present invention comprises a cell culture assembly comprising at least one channel comprising an upstream portion and a downstream portion; at least one cell culture chamber having at least one single-cell attachment surface positioned between the upstream portion and the downstream portion, the single-cell attachment surface comprising a first portion of a stretchable material; a first vacuum channel positioned between the upstream portion of the channel and the single-cell attachment surface, the first vacuum channel comprising a second portion of a stretchable material operatively connected to the first portion of the stretchable material; a second vacuum channel positioned between the downstream portion of the channel and the single-cell attachment surface, the second vacuum channel comprising a third portion of a stretchable material operatively connected to the first portion of the stretchable material; an inlet positioned at the upstream portion of the channel, the inlet being in fluid communication with the channel; and an outlet positioned at the downstream portion of the channel, the outlet being in fluid communication with the channel.

In another aspect, the current invention comprises a cell culture assembly comprising: a bottom layer having an upstream end and a downstream end, the bottom layer comprising a single-cell attachment support area positioned between the upstream end and downstream end, a first channel positioned between the upstream end and the single-cell attachment support area, and a second channel positioned between the downstream end and the single-cell attachment support area; a membrane positioned over the bottom layer covering the first channel, the second channel, and the single-cell attachment support area thereby forming a cell attachment surface; a middle layer comprising at least one wall attached to the bottom layer of the membrane; and a top layer supported by at least a portion of the middle layer comprising an inlet at the upstream end and an outlet at the downstream end, the inlet being in fluid communication with an area above the single-cell attachment surface and the outlet.

### BRIEF DESCRIPTION OF THE DRAWINGS

FIG. 1a illustrates exemplary embodiment of the present device.

FIG. 1b illustrates an additional exemplary embodiment of the present device.

FIG. 2 is an illustration of a Y-shaped channel embodiment of the present device.

FIG. 3 is an illustration of the inventive device employing a plurality of cell culture chambers.

FIG. 4 is an illustration of the inventive device employing a plurality of cell culture chambers arranged in series.

FIG. 5 is an illustration of the inventive device employing a chemical gradient generator.

FIG. 6a is an illustration of the inventive device applying tensile stress to a single cell.

FIG. 6b is an illustration of the inventive device applying shear stress to a single cell.

FIG. 6c is an illustration of the inventive device applying both tensile and shear stress to a single cell.

FIG. 7 is an illustration of one embodiment of the fabrication process for the inventive device.

FIG. 8 is an illustration of shear stress device with controlled flow along a North-South channel.

FIG. 9 is an illustration of human tenocyte response to fluid shear stress.

FIG. 10 is an illustration of human tenocyte response to static and cyclic tensile strain.

FIG. 11 is an illustration of the inventive microfluidic device system.

### DESCRIPTION OF THE PREFERRED EMBODIMENTS

The present invention provides technology for researchers to apply physical forces to and analyze biochemical and biomechanical responses from a single cell alone or in contact with a select group of other cells in a defined configuration. This microfluidic technology can be used to apply this strain or shear stress to single cells either alone or in connection with other cells in a syncytium so that analysis may be constrained to a single cell's response. The novel microfluidic mechanical loading system allows analysis of the response of a single cell to an applied fluid shear stress and tensile strain in the presence or absence of various chemical mediators and is designed to allow for continuous assessment of multiple outcome measures, such as cell viability, reproduction and metabolic activity, cell morphology, extracellular matrix activity, and cell signaling. The device has the ability to introduce, position, culture, and mechanically load a single cell or small group of cells. This high throughput, next-generation device has broad applications in many fields including stem cells, genomics, tissue engineering, pharmacology, regenerative medicine, and biotechnology. The inventive device may be used as a tool used for developing diagnostic tests for cells subjected to strain and flow and pharmacologic agents, simultaneously, and for analyzing differential cell mechanosensitive responses of normal and pathologic cells in medicine.

The novel microfluidic device can apply tensile strain and/or fluid shear stress to a single cell in whole or in part in vitro. In one embodiment, the device includes a single cell culture chamber, stretch platform, and flow and control channels for simultaneous application of shear stress and/or tensile strain. The cell culture chamber may be coated with specific matrices, such as type I collagen, to enhance cell attachment. The device is capable of introducing, positioning, culturing, and mechanically loading a single cell or small group of cells. The cell culture environment may be used to analyze cell spreading, adhesion, viability, morphology, and growth in the device under no strain or flow conditions. The novel device also allows for measurement of cell responses to strain, fluid flow, a combination of strain and flow, and no strain for up to two weeks in culture.

The inventive device can be used to analyze the fluid flow profile and determine the magnitude of applied strain within the microfluidic device. The fluid flow profile within the device channels under various flow rates can be analyzed using motion analysis of fluorescent dyes and beads added to the flow perfusate. The magnitude of applied strain can be determined using texture analysis and image pattern matching techniques of changes in the substrate and adherent cell shape under strained and unstrained conditions. The microfluidic device can be used to generate lower strain and shear values at the monolayer surface and produce more controlled application of strain and/or shear stress regimens with a higher throughput than do macro systems.

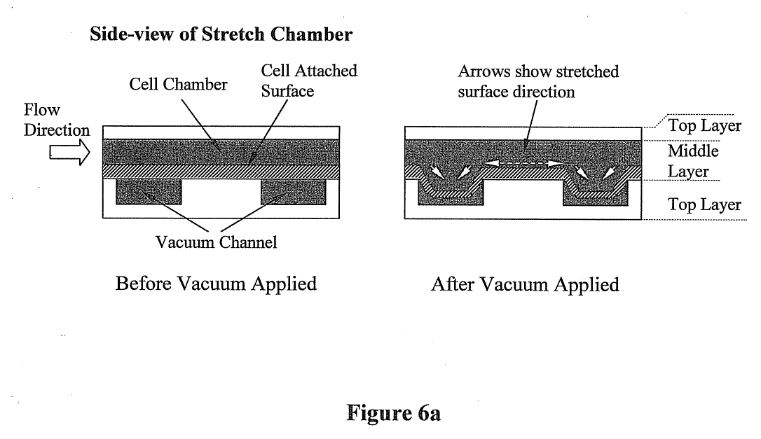

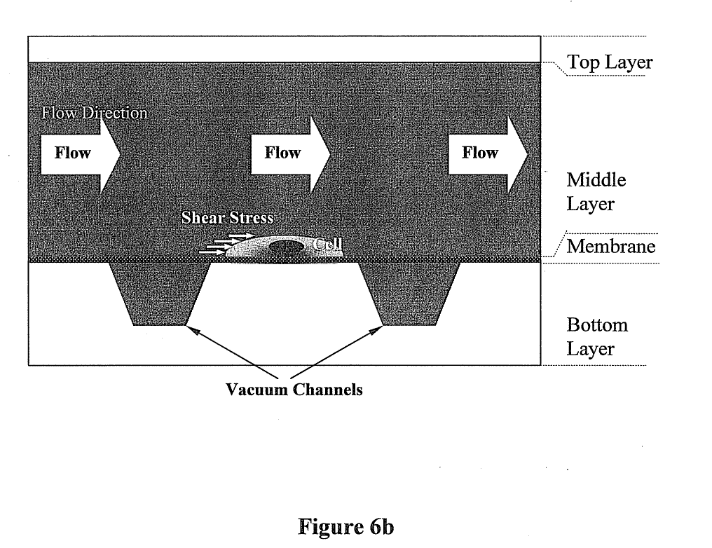

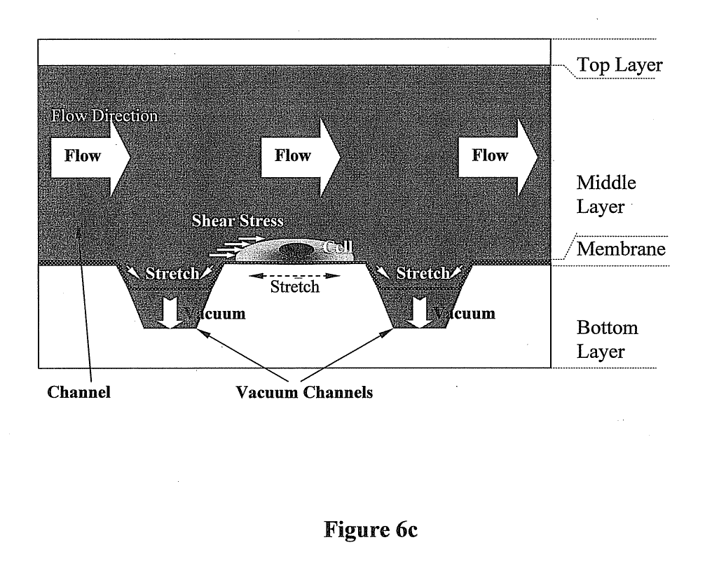

Generally, the inventive device is a microfluidic device. The microfluidic device applies shear stress on a single-cell, cell culture, or tissue. It includes a channel having a cell culture chamber, which has a cell attachment surface. The cell attachment surface is flanked by vacuum channels. See FIG. 6a. Thus, the channel comprises at least two vacuum channels, the first vacuum channel positioned upstream in relation to the cell attachment surface, and the second vacuum channel positioned downstream in relation to the cell attachment surface.

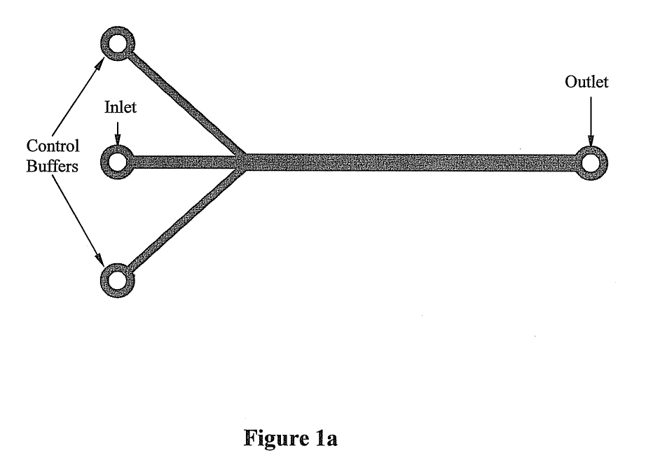

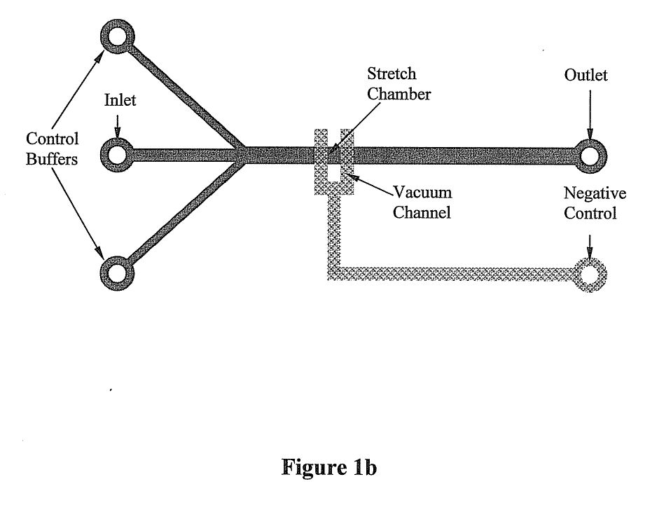

FIGS. 1b, 1c, and 6a illustrate exemplary embodiments of the present device. These figures show the above described channel including an inlet and an outlet. The inlet is generally positioned upstream of the cell attachment surface. A medium may be inserted through the inlet. The medium may flow over the cell attachment surface in the downstream direction. The flow can be regulated by positive pressure applied at the inlet or negative pressure applied at the outlet. The device allows users to apply fluid shear stress to a single cell in culture. Upon reaching the outlet, the medium may be removed from the device and/or may be re-introduced into the inlet. Shear stress can be controlled by controlling the flow rate of the medium. The amount of flow to induce stress on the cell growing in the membrane may be altered and studied. In one embodiment, the membrane may be formed from a transparent material so that the entire assembly may be placed on a microscope. The effect of fluid flow and stress on the cell growing on the membrane may be actively studied.

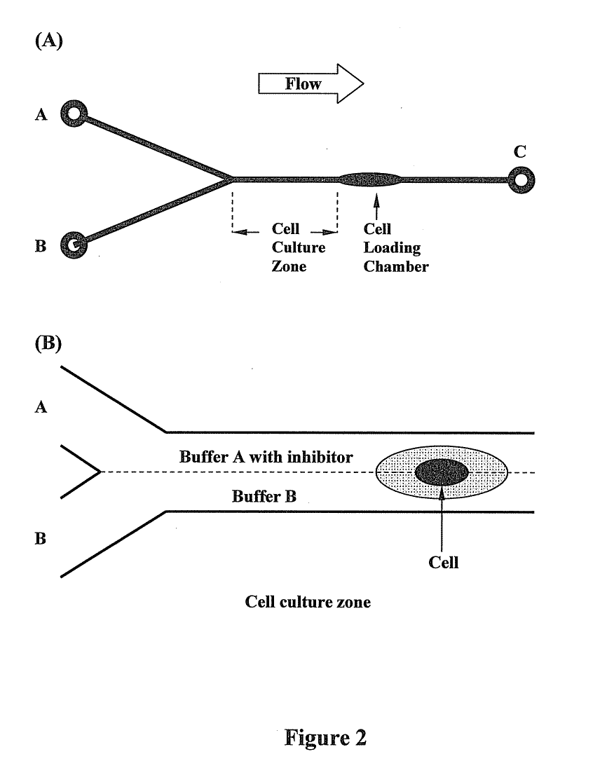

The channel may comprise a plurality of inlets. For instance, a Y-channel and a T-channel as shown may be utilized. However, it should be recognized that the channels may exist in a multitude of configurations. The plurality of inlets may be positioned on the channel or may merge into the channel via inlet channels. In this embodiment, various mediums or liquids can be controlled during the operation of the device. FIG. 2 is an illustration of a Y-shaped channel. These channels are designed for single-cell shear stress response tests. The device includes chemical loading reservoirs, outlet, flow channels, cell culture zone, and cell loading chamber. In one embodiment, positive pressure is applied to reservoirs A and B, the solution will flow from east to west. As explained above, shear stress is controlled by controlling the flow rate.

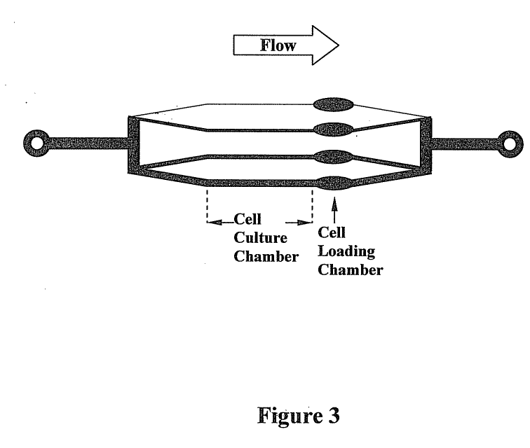

The device includes at least one cell culture chamber having at least one cell attachment surface. Alternatively, the device may have a plurality of cell culture chambers. In one aspect of this embodiment, as illustrated in FIG. 3, the upstream portion of the channel divides into individual cell culture channels, one for each cell culture chamber. At the downstream portion of the channel, each individual cell culture channel merges into the downstream portion of the channel or the outlet. In this aspect, the vacuum channels flank each of the cell culture chambers. Optionally, this aspect may include a plurality of cell loading chambers, one for each individual cell culture channel. The cell chamber width may be varied at the cell culture zones to vary the flow rate and, thus, the applied shear stress.

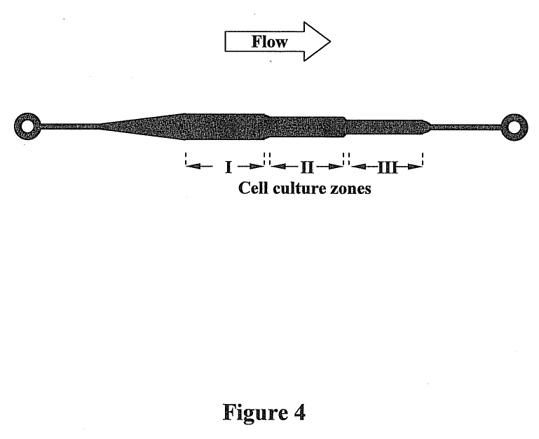

In another aspect of this embodiment, the cell culture chambers are arranged in series. This embodiment is illustrated in FIG. 4. In this aspect, the vacuum chambers may flank each individual cell culture chamber or may flank the cell culture chambers collectively. In this embodiment, the device comprises at least two vacuum channels that flank the cell attachment surface. The vacuum channels are covered by a stretchable membrane that is capable of acting on the cell attachment surface when the membrane is stretched. In one embodiment, the cell attachment surface is a portion of the stretchable membrane between the vacuum channels; therefore, stretching the membrane at the vacuum channels will cause the cell attachment surface to stretch.

The vacuum channels are in communication with a vacuum, whereby the operation of the vacuum creates negative pressure within the vacuum channels. The cell attachment surface comprises a stretchable membrane. The membrane spans the cell attachment surface and the vacuum channels. During operation of the vacuum, the negative pressure within the vacuum channel and/or the positive pressure from the medium stretch the membrane into the vacuum channels. The vacuum is used to deform the membrane applying tensile strain to the cell cultured on the membrane. This action is illustrated in FIGS. 6a and 6c. Consequently, the membrane that spans the cell attachment surface likewise stretches. Tensile stress can be controlled by controlling the force of the vacuum.

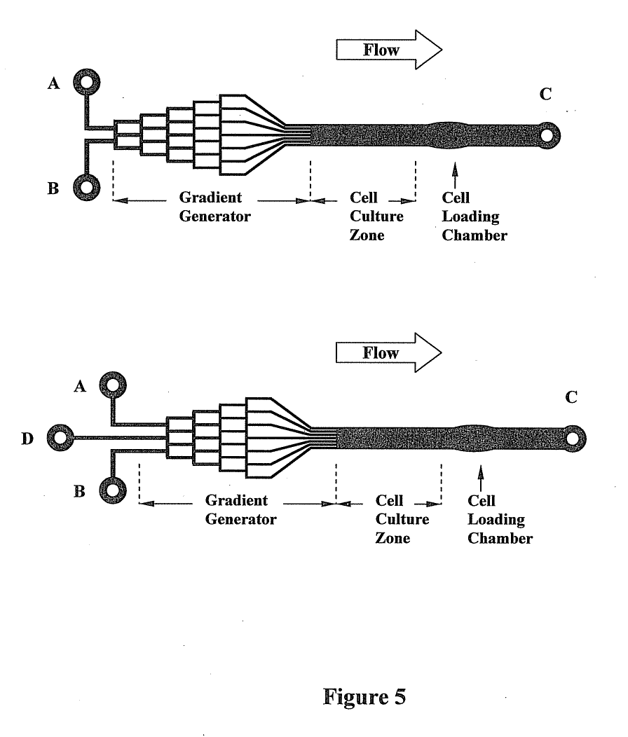

In an additional embodiment, the device includes a chemical gradient generator. This embodiment is illustrated in FIG. 5. The chemical gradient generator is used for mixing two or more chemicals and creating different concentration patterns in the cell culture chamber. This allows treatment of the cells with chemicals during and after the shear stress phase. The chemical gradient generator may be used for mixing two or more chemicals to create different concentration patterns in the cell culture chamber. The upper portion of FIG. 5 illustrates a two-chemical gradient generator, while the lower portion of FIG. 5 illustrates a three-chemical gradient generator device. Here, the channel is divided at the upstream end into a plurality of gradient channels. The plurality of gradient channels has an inlet end and a cell culture zone end. The plurality of gradient channels merges into the channel at the cell culture zone end.

The device may optionally include sensors to monitor culture conditions or cellular responses. Examples of sensors that can be used include sensors for oxygen, carbon dioxide, key metabolities, pH, pressure, flow rate, and microbial contamination. Optionally, the data from the sensors can be displayed during the operation of the device.

One embodiment of the microfluidic device has a bottom layer, membrane, middle layer, and top layer. See, for example, FIGS. 6a, 6b and 6c. The bottom layer forms the base of the device and comprises the vacuum channels. The vacuum channels are wells positioned around a loading post which supports the cell attachment surface. The membrane is positioned over the vacuum channels and the loading post, thereby forming the cell attachment surface. The middle layer comprises a plurality of walls that connect the top layer to the bottom layer. The walls create a height between the membrane and the top layer large enough to permit a desired volume of medium to flow through the device. In one embodiment, the height, measured from the cell attachment surface to the top layer, is from about 60 μm to about 150 μm. The top layer comprises an inlet and an outlet discussed above. FIG. 6c illustrates the operation of the inventive device with the induction of both shear stress (flow of the fluid through the channel) and tensile stress (application of the vacuum around the loading posts supporting the cell attachment surface). As illustrated, the presently described inventive device allows the simultaneous application of shear and tensile strain to single cells.

Another embodiment of the microfluidic device includes a body having an interior space. The interior space includes at least two vacuum channels, a loading post positioned between the first vacuum channel and the second vacuum channel, and a stretchable membrane. The stretchable membrane is positioned over the two vacuum channels and the loading post. The device includes an inlet and an outlet that are in fluid communication with the interior space of the device. In one aspect of this embodiment, the interior space is divided into a plurality of channels, each channel being in fluid communication with the inlet and the outlet. Each channel includes at least one loading post, and at least two vacuum channels positioned on each side of each loading post. Optionally, the outlet may be in fluid communication with the inlet or the interior space near or at the inlet.

The inventive device is designed to maintain a stable temperature. In one embodiment, temperature control is accomplished by incorporation of a heated liquid layer into the microfluidic device. A thermal apparatus outside the system heats a liquid that is pumped into certain channels of the device, thus keeping the entire device at a controlled temperature. In an alternative embodiment, a thermal controller may be integrated into the system.

In one embodiment, a microscale air pressure control system controls tensile strain and shear stress. In this embodiment, a low flow rate pump, such as a Pump 11 Pico Plus syringe pump (Harvard Apparatus, MA), is used for controlling shear stress as these syringe pumps are designed for delivering flow rates from about 2 nl/min to about 440 μl/min with 3-10 mL syringes. Control of fluid flow is achieved by the regulation of the pump flow rate. The flow rate may range from picoliters to milliliters of total flow to continuous flow of fluid. Alternately, the pump flow rate may be maintained at a constant rate. Alternatively, the pump may be used to provide flow reversals so that fluid enters the chamber from one direction at one instant then reverses direction and enters from the opposite side of the chamber at the next instant. These levels of flow control permits both continuous fluid flow, and discontinuous fluid flow, the latter as a pulsating flow or a flow reversal. The precise nature of the rate of fluid flow, and type of fluid flow may have unique consequences for the response(s) of the cells or tissue experiencing the deformation. This is particularly true when fluid flow is combined with substrate strain.

In one embodiment of the operation of the inventive device, fluid is supplied to the channels into the cell culture chamber upstream portion, over the single-cell attachment surface and on to the downstream portion. The cell culture chamber thereby is a flow chamber through which fluid may flow through. A cell is placed on the cell attachment surface and positioned within the flow shafts. Cells may be cultured directly on the cell attachment surface. Cells cultured on the attachment surface are subject to shear stress when fluid flows through the chamber. Negative or positive pressure is applied by the vacuum. In one embodiment, several cell culture chambers are present. The flow rate of fluid applied to each chamber may be varied. One or more of the flow chambers may be used at one time. The flow may be continuous in one direction, the flow may be pulsed or the flow may be occasionally or periodically reversed as described above. In this manner, a variety of stresses may be applied cells grown side-by-side.

A vacuum and valve system is utilized with the device for applying tensile strain. The vacuum and valve system uses vacuum pressure to stretch a membrane, upon which the cells are plated, over a loading post-type feature within the microfluidic device (see FIGS. 5 and 6a) thus straining the cells. Cells may be subjected to tensile strain before, during or after applying shear stress. The shear stress pump and tensile strain vacuum may be computer regulated.

In one embodiment of the operation of the inventive device, when negative pressure is applied, a vacuum is drawn to the underside of a membrane. The flexible membrane is pulled in the direction of the vacuum, i.e., downwardly. In so doing, the membrane is stretched, resulting in an equibiaxial strain on the flexible membrane. The cell, which is adhered to the flexible membrane, likewise experience equibiaxial strain. The vacuum may be applied once, intermittently, regularly or in a variety of frequencies, durations, and amplitudes to induce equibiaxial strain on the cell over time.

Surface treatment of the microstructured components improves cell adherence to the desired areas within the microchannels. In an additional embodiment, use of cold O₂ or N₂ gas plasma treatment effectively reduces hydrophobicity of the silicone rubber, though transiently. Wet chemistry treatments may be used to treat the cell attachment surface with matrix peptides such as type I collagen. In another embodiment, the cell attachment surface may be treated with strong acid, then derivatized with proteins to provide cell adherence.

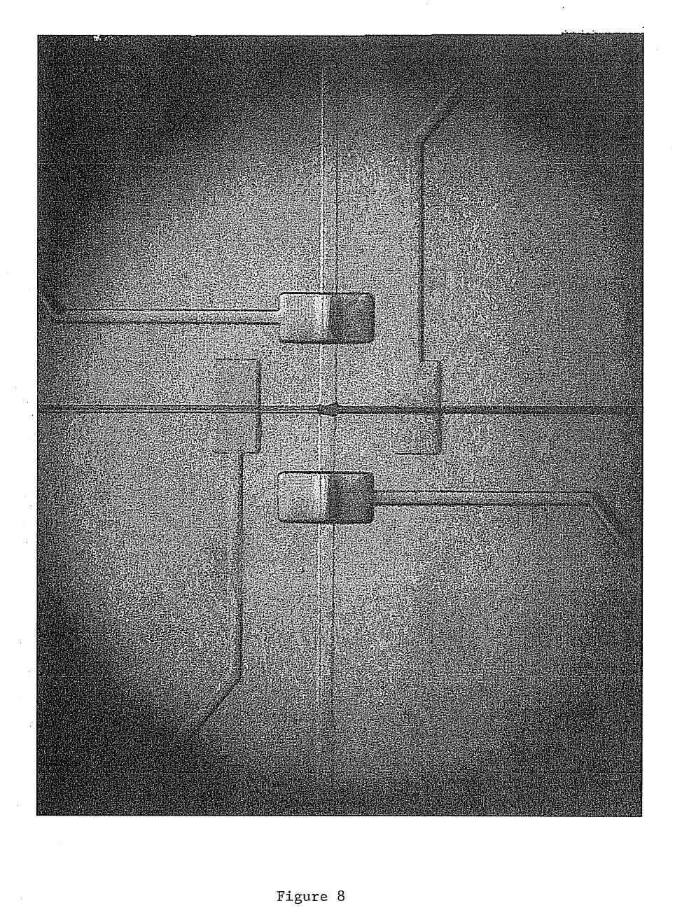

Different microfluidic strategies may be used for positioning single cells in a channel. One method involves single or arrays of dams designed to entrap cells as they move through microchannels by hydrodynamic or electro-osmotic forces. The cell is trapped at a feature that is dimensionally smaller than the cell itself. In the second strategy, single cells are loaded directly into different chambers with static or low flow fluid environments. However, the features of these dams cause certain levels of unpredicted stress on the cells. The microwell format may also be used to trap cells. The microwell method is similar to conventional cell culture methods except cells are cultured in microscale-sized wells. The intersection of the cell channels in the present device is designed for improving cell positioning. See FIG. 8, illustrating a microfluidic shear stress device with controlled flow along the North-South channel. Cells are loaded and cultured in the East-West channel. Valves V₁ and V₃ control shear stress flow, while valves V₂ and V₄ control cell load channel. The North-South channels are coated with an extracellular matrix protein (i.e., collagen). The only extracellular matrix coated area in the cell loading channel, which is the East-West channel, is at the cross section with the North-South channel. Thus, cell attachment at this cross section is selectively increased. Cells are loaded from the West end inlet of the East-West channel. The channel may be washed, for example, for 30 to 60 minutes after cell loading with standard culture media. The cell(s) will attach primarily at the cross section. Fluid flow is applied from the North inlet of the North-South channel and strain applied directly beneath the cross-section.

In an additional embodiment, a single cell may be positioned by a “dam” trap or at the channel intersection when a low concentration of cells is loaded into the cell culture chamber. Additionally, small groups of cells may be positioned and a single cell selected from the group for testing. With a controlled positive pressure, a group of cells, for example, 1-30 cells, may be loaded into the cell culture chamber. Any cells that do not have physical connections with other cells may be selected for shear stress experiments. A similar strategy is utilized with single cell stretch procedures. More than one cell may be stretched on the micro-stage. Materials suitable for the construction of the microchip are well known in the art. Suitable materials include silicone elastomer surfaces. Type I collagen peptides may be used to coat channels.

The fluid flow profile within the device under various flow rates may be analyzed by various methods. In one embodiment, flow rates may be determined using motion analysis of fluorescent dyes and beads added to the flow perfusate. The magnitude of applied strain is determined using texture analysis and image pattern matching techniques of changes in the substrate and adherent cell shape under strained and unstrained conditions. The microfluidic device may be used to generate lower strain and shear values at the monolayer surface, better control and response times, and higher throughput than do macro systems.

The present invention allows for flow rate calculation so a given shear stress in the cell culture chamber is achieved. Fluid flow within a microfluidic device is considered to be laminar, thus the following equation for wall shear stress can be applied:

μ=6μQ/bh²

where μ is the shear stress in dynes/cm², μ is the viscosity of the fluid in dynes/cm², Q is the flow rate in ml/s, b is the width of the flow channel in cm, and h is the height of the flow channel in cm. To analyze the fluid flow profile within the device under multiple shear stresses, polystyrene microspheres, such as FluoSpheres® (Molecular Probes), and fluorescent dyes (i.e., rhodamine) may be added to the perfusate and the flow stream imaged using a fluorescence microscope, such as an Olympus BX60 fluorescence microscope and image analysis system. The flow profiles may also be analyzed in the presence and absence of cyclic and static strain.

Cell strain magnitudes may be determined with a custom, texture correlation, strain analysis program written in Matlab language and used as previously described. Briefly, images of fluorescently labeled cells before and after a given strain level are filtered with a two-dimensional Wiener filter, and then bi-cubically interpolated at ⅓ pixel to increase pixels. A grid of user-defined nodes is placed over a region of interest on an interpolated image of non-strained cells. The program then performs a two-dimensional normalized cross-correlation to determine which area of the image of strained cells best matches the pixel pattern of the non-strained image template, a square area of pixels around each node. A zero-order approach, which assumes that the template remains square, is utilized to determine the displacement of the pixels in the template. The center of the pixel pattern that returns the greatest correlation coefficient is determined as the displaced node on the strained image. The coordinates of the displaced nodes is used to calculate strain with a strain-displacement matrix computed for a four-node quadrilateral element. Grid and element sizes are chosen such that nodes will be distributed solely over the cell of interest. Template sizes are chosen to maximally cover an area around a node without returning an error. Displacements between nodes are assumed to vary linearly within each element. Strain magnitudes are calculated with the Lagrangian finite strain tensor for a continuum body.

To verify the cell strain values computed with the texture correlation program, manual line measurements may be made in each cell using the measuring tool, such as the tool in Photoshop®. In a non-limiting example, three line measurements may be made in both directions at the same location within the same cell and averaged. These measurements may be taken at the same “landmark” point in the non-strained image and the first image after the onset of substrate strain for all cells analyzed by texture correlation. Engineering strain (δl/l) is then used to calculate strain with all manual measurements.

It will be readily appreciated by those skilled in the art that modifications may be made to the invention without departing from the concepts disclosed in the foregoing description. Such modifications are to be considered as included within the following claims unless the claims, by their language, expressly state otherwise. Accordingly, the particular embodiments described in detail herein are illustrative only and are not limiting to the scope of the invention which is to be given the full breadth of the appended claims and any and all equivalents thereof.

### EXAMPLES

### Example 1

### Fabrication of the Microfluidic Device

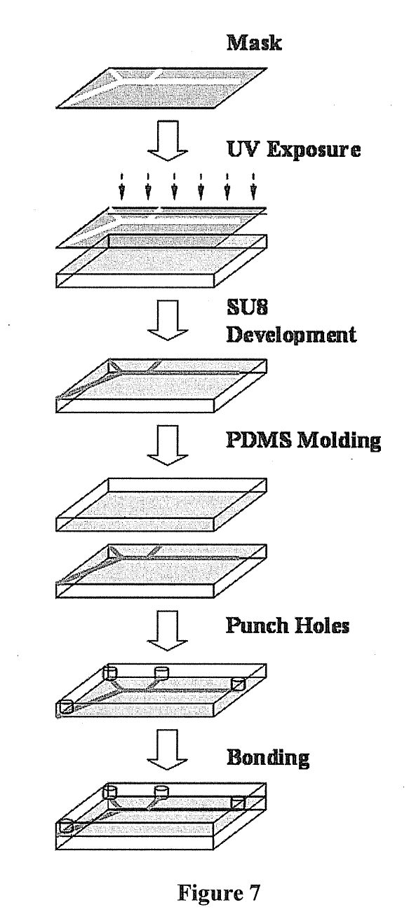

The microfluidic device is designed with TurboCAD and Solid Edge™ CAD programs and then fabricated in polydimethylsiloxane (PDMS) using soft lithography and replica molding. FIG. 7 illustrates one embodiment of this fabrication process. A master with positive relief patterns of cell culture chambers and the control system is created using photolithography. Two layers of negative photoresist, SU-8, are spun-coated onto a 3-inch silicon wafer. Ultraviolet radiation is used to create microstructures by projecting the shadows produced by a working mask onto the light-sensitive resist. Mercury arc lamps with 365 nm UV emissions are used as an exposure light source. The SU-8 developer is then used for developing the photoresist. Before pouring the PDMS over the mold, the SU-8 mold is silanized in a vacuum desiccator cabinet for 1 hour. A prepolymer mixture of Sylgard 184 (Dow Corning) is then cast and cured against the positive relief. PDMS pieces are then sterilized with 70% ethanol and dried by blowing the surface with nitrogen gas. Cleaned glass microslides (51×51 mm, No. 1, Corning Inc.) are immersed in a sterile aqueous solution of 1.0 mg/ml poly(l-lysine) (PLL, M.W. 70,000-150,000; Sigma) in borate buffer for 24 hours before use. Sealing the PDMS piece to a polylysine-coated glass coverslip by conformal contact will form the enclosed channels. This type of reversible contact results in both a water-tight seal during use and allows for the separation of the PDMS from the glass cell culture surface after the experiment for further processing if necessary. For making permanent bonded chips, oxygen plasma treatment is used. The surfaces of glass and/or PDMS pieces are treated with 50 W of RIE power for 30 seconds.

A typical microchip includes a micro/macro interface, liquid transport channels, and a cell culture chamber, all designed for connecting the microchip to the macro devices such as the pump. This interface includes inlet and outlet reservoirs, tubing, and adaptors. Transport channels are used for delivering liquid, chemicals, and cells. These channels are from about 80 μm to about 120 μm wide and from about 150 μm to about 500 μm high. The applied pressure for moving fluid and cells through the channels is from about 4 psi to about 5 psi. For moving air/hydraulic pressure or vacuum through the control channel from about 15 psi to about 18 psi is applied. A cell culture chamber ranging from about 45 μm to about 500 μm in width is used for culturing a cell or small group of cells. Cell traps are designed for loading a cell at a selected position in the channel. A micromixer for on-chip fluorescence labeling and a gradient generator for selective chemical distribution to the cell culture chamber are included.

### Example 2

### Cell Culture, Loading, Introduction, and Positioning of Cells in Microfluidic Channels

MC3T3-E1 mouse osteoblast-like cells are cultured in 100 mm diameter dishes with complete serum-containing Dulbecco's Modified Eagle Medium (DMEM) for 7 days, then released from the culture dishes with trypsin-EDTA. Cells are collected and sedimented by centrifugation. Cell pellets are filtered with a 50 μm nylon screen. Cells are loaded into the microfluidic device at 3×10⁶ cells/ml (10 ml volume) yielding approximately 100 cells in the cell chamber. Cells are cultured 3 to 7 days after loading into the microchannels. Media is changed with a slow flow media driven by a syringe pump every two days.

Cells are loaded into the device using the following method: 1) all valves are closed and the syringe tubing inserted into the cell loading inlet; 2) providing minimal pressure to the syringe top, the cells are injected while viewing the channel under a microscope to visualize the cells flowing into the channels; and 3) once the cells are loaded, both the inlet and outlet channels to the cell loading chamber are closed.

### Example 3

### Cell Spreading, Adhesion, Viability, Morphology, and Growth

MC3T3-E1 osteoblast-like cells are loaded into the microfluidic device, positioned, and cultured for up to two weeks. Cells are imaged daily to visualize cell morphology as well as monitor cell movement and growth within the chamber. Monitoring is accomplished with an Olympus BX60 microscope. After two weeks, cell viability is determined using the LIVE/DEAD® Viability/Cytotoxicity Kit for mammalian cells (Molecular Probes/Invitrogen, Carlsbad, Calif.). This kit utilizes the fluorescent dyes calcein AM, to label live cells green, and ethidium homodimer, to label dead cells red. After labeling, the cells within the device are visualized with a Olympus BX60 fluorescence microscope. Images are collected by F-View Soft Imaging system with MicroSuite Biological Suite software. Cells are also fixed in 3.7% formaldehyde after two weeks of culture and labeled with 4′, 6-diamidion-2-phenylindole (DAPI) and rhodamine phalloidin to visual the nucleus and actin cytoskeleton, respectively.

### Example 4

### Device Functionality

After positioning and culturing cells within the device, cells are then subjected to four different dose-controlled fluid shear stress and tensile strain regimens: 1) static culture within the device (control); 2) fluid shear stress alone at 1, 5, and 10 dynes/cm²; 3) tensile strain at 1%, 3%, and 5% static strain and at 1 Hz; and 4) combined stimulation with fluid shear stress and tensile strain. Cells are then analyzed for changes in [Ca²⁺]_{ic} before, during, and after loading. Mouse osteoblast-like or mouse cardiovascular endothelial are used for these experiments. ATP-induced Ca²⁺ signaling of the cells is used as a positive control. Additionally, cell responses to identical regimens applied with macro-devices are used for comparison. Additionally, on chip cell staining procedures for actin and the nucleus are used to monitor morphologic changes.

Method 1: Application of tensile strain and fluid shear stress at the macrolevel. Cells are cultured on membranes under regulated tensile strain in micromass spots at 2000 cells/10 μl, grown to quiescence and used on day 6 of culture. Calcium responses of the cells subjected to the individual mechanical stimulations and in combination (with regulated fluid flow) are compared to those Ca²⁺ responses of the cells in the microfluidic device. This experiment focuses on comparing responses of a single-cell with mass cultured cells after shear stress. Low and high shear stress or stretch experiments are performed. This initial data generated from single cell experiments not only provides valuable data for understanding cells' responses to mechanical stimuli, but also generates data for shear stress and stretch spontaneously.

Method 2: Intracellular Ca²⁺ imaging. Cells are analyzed for changes in [Ca²⁺]_{ic} in response to fluid shear stress and tensile strain. Cells are rinsed with Earles' Balanced Salt Solution (EBSS), incubated at room temperature in 5 μM Fura-2AM (Molecular Probes) with 0.1% Pluronic F-127 (Molecular Probes) and 0.5% DMSO for 60 minutes at room temperature, then rinsed with EBSS. An upright fluorescence microscope, equipped with a 40× water immersion ultraviolet objective lens, a Sutter Lambda DG4 wavelength switcher and light guide (Novato, Calif.), and a CoolSnap digital camera (Roper Scientific, Trenton, N.J.), are used to assess [Ca²⁺]_{ic} using the ratio dye method and image analysis software (ISee Imaging Systems, Raleigh, N.C.). Baseline [Ca²⁺]_{ic} is quantified for 60 seconds prior to stimulation. All cells with an average increase in [Ca²⁺]_{ic} three standard deviations over its basal level are considered to have elevated its [Ca²⁺]_{ic}.

Method 3: On chip cell staining. In addition to cell signaling, mechanical loading can affect other cell activities such as attachment, migration, orientation, and proliferation. Therefore, stained cells within the microchannels may be used to observe these other cell behaviors. Cells are grown for 3-5 days then serum starved for 24 hours before starting experiments. Cells are subjected to an applied shear stress for 24 to 72 hours. Cells are fixed with 3.7% formaldehyde in phosphate-buffered saline (PBS), pH 7.2, at room temperature for 15 min, permeabilized with 0.1% Triton X-100 in PBS at room temperature for 20 min, and rinsed twice with PBS. Actin filaments (microfilaments) are stained at room temperature for 1 hour with rhodamine phalloidin (Molecular Probes, Eugene, Oreg., at 1:400 dilution in PBS). Cells are then be rinsed with PBS twice and visualized with a fluorescent microscope.

DAPI is used for dye exclusion tests following the method of Baskin et al. (2003) to detect nonviable cells with compromised membranes in live cell cultures. Briefly, cells are incubated with 200 ng/ml DAPI (Sigma, St. Louis, Mo.) in cell culture medium at 20° C. for 30 minutes prior to each addition of thimersol at 45 minutes and 2, 4, 6, and 24 hours. A fluorescence signal is monitored, and representative images acquired at each time point using an Olympus BX-60 fluorescent microscope equipped with a F-View digital camera system (Soft Imaging System GmbH) and image analysis software (Olympus MicroSuite Biological Suite).

### Example 5

### Tendon Cell Response to Fluid Flow

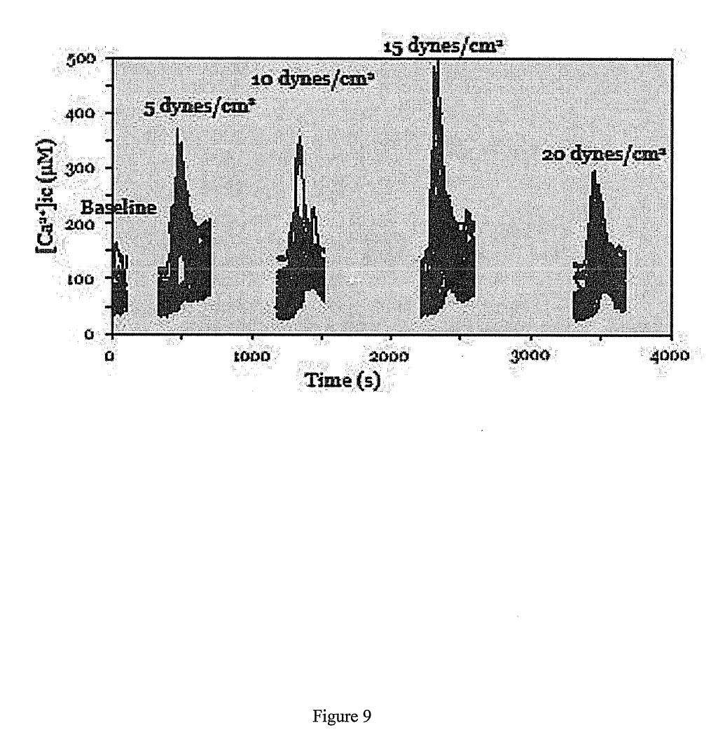

Tendon cells respond to fluid flow by increasing [Ca²⁺]_{ic}. Tenocytes were subjected to shear stresses of 0, 5, 10, 15, and 20 dynes/cm². Tenocytes were subcultured onto collagen peptide bonded glass cover slips at 25,000 cells/cm² and grown to quiescence. Cells were washed in EBSS then loaded with 5 μm Fura-2AM in 0.1% Pluronic-127 in EBSS for 2 hrs. Cells were washed with EBSS and the cover slip transferred to the top of the FlexFlow™ chamber. A Masterflex pump was used to deliver flow rates calculated to provide 5, 10, 15, and 20 dynes/cm² at the monolayer surface. Flow experiments were conducted in EBSS with and without Ca²⁺. The average single-cell [Ca²⁺]_{ic} for all cells in a field was determined prior to the initiation of flow and five minutes following flow induction which allows determination of the shift in baseline [Ca²⁺]_{ic}. Calcium transients were defined as those [Ca²⁺]_{ic} which increased by 100 nM over basal levels. See FIG. 9.

Human tenocytes at rest had a single cell basal [Ca²⁺]_{ic} of 40-80 nM. Seventy to 83% of cells increased their [Ca²⁺]_{ic} to a transient level of over 200 mM within 25 to 60 seconds after initiating flow, which gradually decreased over the next 2-4 minutes (FIG. 9). Cells did not increase [Ca²⁺]_{ic} in response to flow-induced shear stress in Ca²⁺-free EBSS. Human tenocytes respond to fluid-induced shear stress in vitro by signaling with an increase in [Ca²⁺]_{ic}. The shear stress-induced increase in [Ca²⁺]_{ic} was dependent upon extracellular Ca²⁺ suggesting a role for Ca⁺ channels in the plasma membrane during mechanotransduction.

### Example 6

### Tendon Cell Response to Shear Strain

Tendon cells respond to tensile strain by increasing [Ca²⁺]_{ic}. Tendons are constantly subjected to mechanical load such as shear stress and strain during day-to-day activities. This experiment was designed to test the hypothesis that human tendon surface cells would respond to mechanical stretching by increasing [Ca²⁺]_{ic} through multiple pathways. To evaluate the Ca²⁺ response, tenocytes were spot cultured at 2,000 cells/10 μL in the middle of a flexible silicone membrane and grown to quiescence. On the sixth day after culture, the cells were rinsed with EBSS with HEPES, pH 7.2, Ca²⁺ and Mg²⁺, incubated at room temperature in 5 μM Fura-2AM for 90 minutes, then rinsed with EBSS. The membranes were transferred to a device that applies an equibiaxial strain to the cells across a 25 mm loading post. The unit was mounted on the stage of an Olympus upright fluorescence microscope to permit assessment of [Ca²⁺]_{ic} using a ratio dye method. Baseline Ca²⁺ was quantified at no stretch conditions then strain was applied either statically (1%, 2%, 4%, and 6% elongation, for 1 min) or cyclically (0.1 Hz, 1%, 2%, 4%, and 6% elongation, for 1 min).

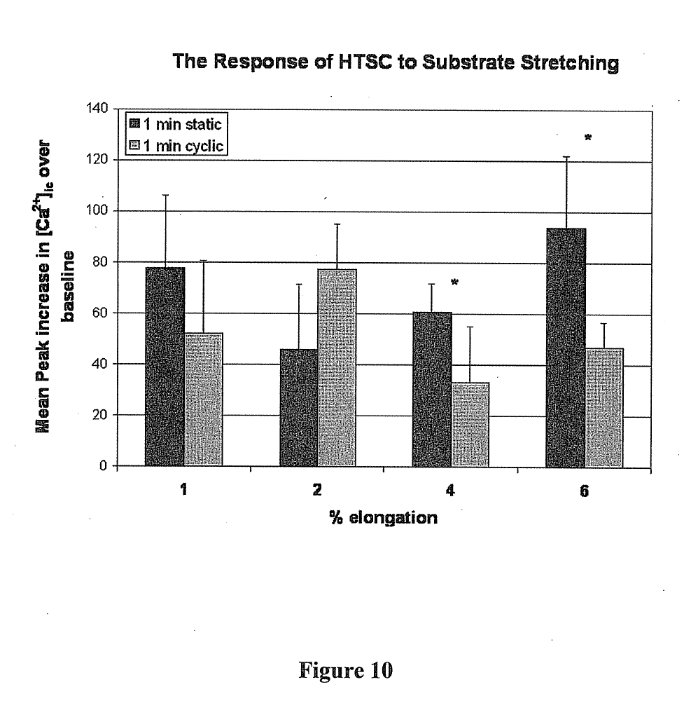

Tenocytes responded to mechanically induced strain by increasing [Ca²⁺]_{ic}. The response to static stretching was two-fold greater than to cyclic stretching for the 4% and 6% elongation (p<0.05; see FIG. 10). There was no significant difference at the lower strains between the responses to static and cyclic stretching. The response to mechanical load of tenocytes involves a variety of different pathways and chemical mediators. Unlike a response to fluid-induced shear stress, the stretch response was not dependent upon extracellular Ca²⁺. Furthermore, tenocytes responded to substrate stretching differently as seen by the greater increase in Ca²⁺ signaling with statically stretched cells as compared to cyclically stretched cells. These findings indicate that tenocytes detect and respond to stretch and shear stress in different ways.

### Example 7

### Microfluidic Cell Culture and Shear Stress Device

The purpose of this study was to develop a novel microfluidic device for analyzing single cell responses to fluid flow in the presence or absence of various chemical mediators. A microfluidic device was as described in Example 1. The device was patterned to create a Y-shaped channel designed for controlling fluid flow and chemical distribution (FIG. 1). MC3T3-E1 osteoblasts were suspended in DMEM at 2×10⁶ cells/ml. Ten μl of the cell suspension were loaded into the cell culture chamber of the microfluidic device. Cells were then perfused with EBSS using a syringe pump for 4 hours at a shear stress of 0.5-2 dynes/cm². Cells were then fixed with 3.7% formaldehyde in the chip and stained with DAPI and rhodamine phalloidin to label nuclei and actin, respectively.

Drugs can be precisely applied to selected parts of a cell surface with the Y-type channel junction and focusing channels (FIG. 1). MC3T3-E1 cells adhered, spread, and aligned head-to-tail in the cell bioreactor channels. Cells in a 500 μm wide microchannel showed normal morphology. Cells began aligning in the fluid flow direction after 4 hours of perfusion at 2 dynes/cm².

This study reports successful fabrication of a microfluidic device in which cells can be cultured and subjected to fluid shear stress. Additionally, the device allowed for focusing of a chemical within the cell culture chamber of the device. Thus, this device could be used to apply fluid shear stress and/or chemical mediators to subcellular sections for analyzing cell signaling responses. Cells began to align in the direction of flow by 4 hours, but not all cells were aligned in the channel. Longer perfusion times and/or higher shear stresses may be necessary to get all cells to align. This single cell based microfluidic device may be a useful tool in understanding cellular responses to mechanical load and the pathways involved in mechanotransduction.

### Example 8

### Application of Shear Stress to Single Cells

The purpose of this study was to develop a microfluidic device for on-chip shear stress application and analysis of single-cell reactions with fluorescence markers under a controlled mechanical microenvironment.

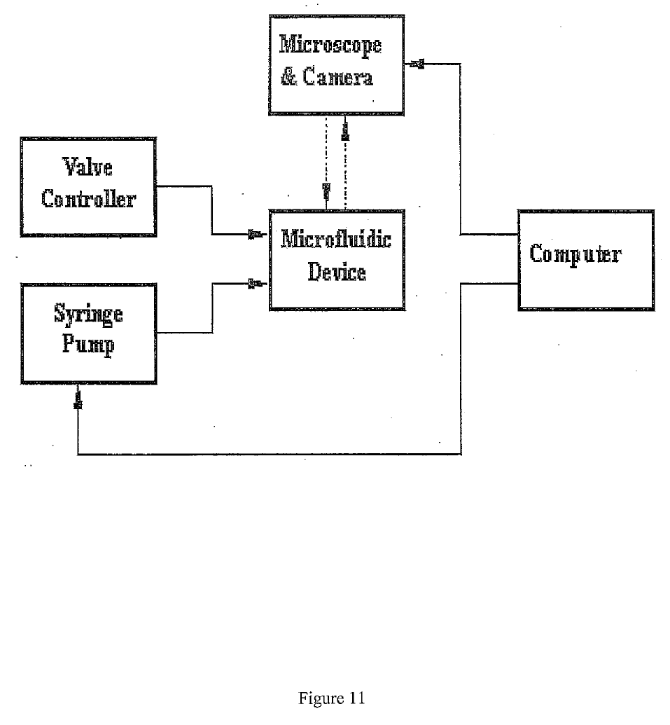

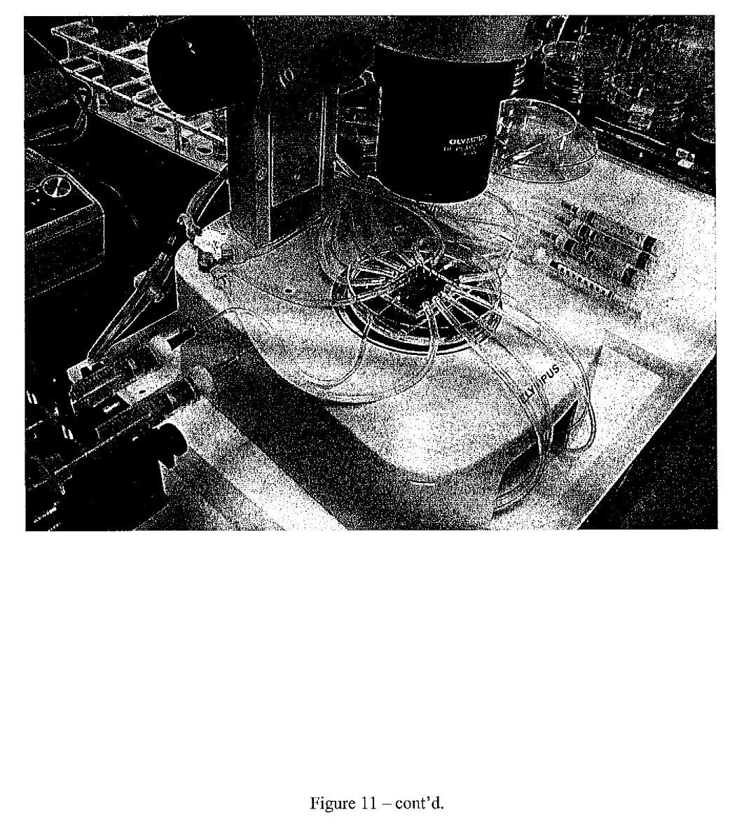

The microfluidic device (FIG. 11) is a multi-layered PDMS structure built using soft lithography and replica molding techniques. The transport microchannels were 80 μm wide by 150 μm high by 2 mm long, generating a channel volume of 24 nl. The cell culture chamber widths were 45, 100, 150, and 200 μm, and the height was the same as the transport channels. Fluid and cell delivery were facilitated by a syringe pump at flow rates between 20-40 μl/min. Human tenocytes were utilized in this study and maintained in Medium 199 with 20% FBS. Ten μl of the cell suspension at 4×10⁵ cells/ml were loaded into the cell culture chamber. After culturing for 3-7 days, cells were perfused with EBSS for 4 hours at a shear stress of 2 dynes/cm². Cells were then fixed with 3.7% formaldehyde in the chip and stained with DAPI and rhodamine phalloidin to label nuclei and actin, respectively.

Single or low number groups of human tenocytes adhered and spread in the microchannels. Cells cultured in a microchannel up to 7 days showed normal fibroblast morphology. A selected shear stress could be precisely applied to the cell surface in the microchannel. After 4 hours of fluid shear stress at 2 dynes/cm², the cytoskeleton was reorganized. Lamellipodia retracted, and organized, robustly stained actin fibers were apparent at the cell periphery, especially facing the fluid flow direction.

This experiment demonstrates the design and development of a microfluidic device in which single tenocytes can be cultured and subjected to fluid shear stress. Additionally, the device allowed for optical analysis of the tenocytes during and after mechanical loading. The cells were fluorescently labeled and visualized without interference from background fluorescence. Staining of the cells within the culture chamber indicated that tenocytes responded to the fluid shear stress by rearranging their actin cytoskeleton. This device could be used to apply various shear stress regimens in the presence and/or absence of chemical mediators to analyze the responses of a single cell to changes in its microenvironment. As a research tool, this single-cell based microfluidic device may be useful in understanding cellular responses to mechanical load and the pathways involved in mechanotransduction.

### Example 9

### Application of Shear Stress to Fibroblasts

Fibroblasts increase intracellular calcium in response to applied shear stress. Application of fluid shear stress can include laminar flow, oscillating flow, or flow reversals delivered to cells in a syncytium. The objective of this study was to address the mechanosensitivity of different segments of single cells to laminar flow in a novel microfluidic chamber.

Soft lithography technology was used to develop a PDMS shear stress microchip that contained a cell culture chamber and four shear stress channels. Several dimensional iterations for the cell chambers were made, ranging from 20, 45, 60, and 90 μm in width, and 60 μm in height. Thirty minutes after loading MC3T3-E1 osteoblast-like cells into the cell chamber, the chip was connected to a perfusion system including low flow rate pump, such as a Pico Plus Syringe Pump (Harvard Apparatus, MA), at a flow rate of 0.1 μl/min for 5 minutes transport tubing and a 3-ml syringe filled with DMEM media in a CO incubator for overnight culture. Real-time monitoring of cell behavior under fluid shear was demonstrated by observing intracellular calcium using the fluorescent dye Fura-2AM.

MC3T3-E1 cells adhered, spread, and aligned head-to-tail in the cell bioreactor channels. Cells in the 45 μm width channel showed normal morphology compared to controls. One of the four shear stress channels was selected for applying a controlled shear stress to a cell pseudopod. The target cell increased [Ca²⁺]_{ic} as shear stress increased from 0.5 to 10 dynes/cm².

The invention claimed is:

1. A cell culture assembly comprising:
at least one channel comprising an upstream portion and a downstream portion;
at least one cell culture chamber having at least one single-cell attachment surface positioned between the upstream portion and the downstream portion, the single-cell attachment surface comprising a first portion of a stretchable material;
a first vacuum channel positioned between the upstream portion of the channel and the single-cell attachment surface, the first vacuum channel comprising a second portion of a stretchable material operatively connected to the first portion of the stretchable material;
a second vacuum channel positioned between the downstream portion of the channel and the single-cell attachment surface, the second vacuum channel comprising a third portion of a stretchable material operatively connected to the first portion of the stretchable material;
an inlet positioned at the upstream portion of the channel, the inlet being in fluid communication with the channel; and
an outlet positioned at the downstream portion of the channel, the outlet being in fluid communication with the channel.

2. The cell culture assembly of claim 1, wherein the at least one single cell culture surface is coated with an extracellular matrix protein.

3. The cell culture assembly of claim 2, wherein the extracellular matrix protein is type I collagen.

4. The cell culture assembly of claim 1, wherein the cell attachment surface is a portion of the stretchable material between the vacuum channels.

5. The cell culture assembly of claim 1, further comprising a plurality of cell culture chambers.

6. The cell culture assembly of claim 5, wherein the plurality of cell culture chambers are arranged in a series.

7. The cell culture assembly of claim 1, further comprising a chemical gradient generator.

8. The cell culture assembly of claim 1, further comprising one or more sensors to monitor culture conditions.

9. The cell culture assembly of claim 8, wherein the sensors are selected from the group consisting of temperature, pH, pressure, flow rate, and microbial contamination sensors.

10. The cell culture assembly of claim 1, wherein the culture conditions are thermally controlled.

11. The cell culture assembly of claim 10, wherein the culture conditions are thermally controlled by incorporation of a heated liquid layer.

12. The cell culture assembly of claim 1, further comprising a vacuum system.

13. A cell culture assembly comprising:
a bottom layer having an upstream end and a downstream end, the bottom layer comprising a single-cell attachment support area positioned between the upstream end and downstream end, a first channel positioned between the upstream end and the single-cell attachment support area, and a second channel positioned between the downstream end and the single-cell attachment support area;
a membrane positioned over the bottom layer covering the first channel, the second channel, and the single-cell attachment support area thereby forming a cell attachment surface;
a middle layer comprising at least one wall attached to the bottom layer of the membrane; and
a top layer supported by at least a portion of the middle layer comprising an inlet at the upstream end and an outlet at the downstream end, the inlet being in fluid communication with an area above the single-cell attachment surface and the outlet.

14. The cell culture assembly of claim 13, wherein the single cell culture chamber is coated with an extracellular matrix protein.

15. The cell culture assembly of claim 14, wherein the extracellular matrix protein is type I collagen.

16. The cell culture assembly of claim 13, wherein the cell attachment surface is a portion of the stretchable material between the vacuum channels.

17. The cell culture assembly of claim 13, further comprising a plurality of cell culture chambers.

18. The cell culture assembly of claim 17, wherein the plurality cell culture chambers are arranged in a series.

19. The cell culture assembly of claim 13, further comprising a chemical gradient generator.

20. The cell culture assembly of claim 13, further comprising one or more sensors to monitor culture conditions.

21. The cell culture assembly of claim 20, wherein the sensors are selected from the group consisting of pH, pressure, flow rate, and microbial contamination sensors.
